(19) United States
(12) Patent Application Publication
(10) Pub. No.: US 2024/0242351 A1
(43) Pub. Date: Jul. 18, 2024

(54) MEDICAL IMAGE PROCESSING APPARATUS, METHOD, AND MEDIUM

(71) Applicant: TERUMO KABUSHIKI KAISHA, Tokyo (JP)

(72) Inventors: Kohtaroh KUSU, Ebina Kanagawa (JP); Yuki SAKAGUCHI, Fujisawa Kanagawa (JP)

(21) Appl. No.: 18/620,980

(22) Filed: Mar. 28, 2024

Related U.S. Application Data
(63) Continuation of application No. PCT/JP2022/036100, filed on Sep. 28, 2022.

(30) Foreign Application Priority Data
Sep. 29, 2021 (JP) 2021-160020

(51) Int. Cl. G06T 7/00; G06T 5/50; G06T 7/11; G06T 15/00; G06V 10/764

(52) U.S. Cl. CPC G06T 7/0012; G06T 5/50; G06T 7/11; G06T 15/00; G06V 10/764; G06T 2207/20221; G06T 2207/30021; G06T 2207/30096

(57) ABSTRACT

An image processing apparatus for processing images of a luminal organ includes a first circuit connectable to a catheter having an ultrasonic probe and insertable into the organ, a second circuit connectable to a display, and a processor configured to: control the catheter to acquire cross-sectional images of the organ when the catheter is inserted thereinto and moved along a longitudinal direction thereof, input the images into a learning model and for each image, obtain position data indicating a boundary between regions of the organ based on segmentation data output from the model, select two consecutive images and identify a group of points corresponding to the boundary in each image based on the position data, associate points in one selected image with points in the other image, and display a 3-D image in which the points in one selected image are connected to the points in the other image.

### CROSS-REFERENCE TO RELATED APPLICATION

This application is a continuation of International Patent Application No. PCT/JP2022/036100 filed Sep. 28, 2022, which is based upon and claims the benefit of priority from Japanese Patent Application No. 2021-160020, filed Sep. 29, 2021, the entire contents of which are incorporated herein by reference.

### BACKGROUND

### Technical Field

Embodiments described herein relate generally to a medical image processing apparatus, a method, and a medium.

### Related Art

In the procedure of percutaneous coronary intervention (PCI), interpretation of medical images generated by imaging modalities, such as intravascular ultrasound (IVUS), optical coherence tomography (OCT), and optical frequency domain imaging (OFDI), is difficult, and thus automation for such interpretation has been studied. In addition, diagnosis and treatment of a lesion or the like are performed using imaging and angiography in combination.

There is an image diagnosis apparatus that generates a blood vessel cross-sectional image using an ultrasonic wave emitted from a catheter inserted into a blood vessel to a vascular tissue and reflected thereby.

In order to associate imaging and angiography, it is important to generate an image of a blood vessel that shows an anatomical feature thereof accurately.

### SUMMARY

Embodiments of this disclosure provide a medical image processing apparatus, a method, and a medium capable of generating a 3-D image of a blood vessel that shows an anatomical feature thereof accurately.

In one embodiment, a medical image processing apparatus for processing medical images of a luminal organ comprises a first interface circuit connectable to a catheter having an ultrasonic probe and insertable into the luminal organ, a second interface circuit connectable to a display, and a processor configured to: control the catheter to acquire a plurality of cross-sectional images of the luminal organ when the catheter is inserted into the luminal organ and moved along a longitudinal direction thereof, input the acquired images into a first machine learning model that has been trained to classify each pixel in an image of a luminal organ and for each of the acquired images, obtain position data indicating a boundary between predetermined regions of the luminal organ based on segmentation data output from the first machine learning model that classifies each pixel of the acquired image, select two of the images that are consecutive and identify a group of points corresponding to the boundary in each of the selected images based on the position data, associate one or more of the points in one of the selected images with one or more of the points in the other image, generate a 3-D image of the luminal organ in which said one or more of the points in one of the selected images are respectively connected to the associated points in the other image, and control the display to display the generated 3-D image.

### BRIEF DESCRIPTION OF DRAWINGS

FIG. 1 is a diagram illustrating a configuration of an image diagnosis system according to an embodiment.

FIG. 2 is a diagram illustrating a configuration of an information processing device according to an embodiment.

FIG. 3 is a diagram illustrating a first example of a configuration of a first learning model.

FIG. 4 is a diagram illustrating a second example of the configuration of the first learning model.

FIG. 5 is a diagram illustrating an example of a configuration of a second learning model.

FIG. 6 is a diagram illustrating an example of segmentation data output by the first learning model.

FIG. 7 is a diagram illustrating a method of identifying a corresponding point group of a boundary of a predetermined site in a case where there is no side branch.

FIG. 8 is a diagram illustrating an example of a method of connecting corresponding point groups.

FIG. 9 is a diagram illustrating a first example of a method of identifying a corresponding point group of a boundary of a predetermined site in a case where there is a side branch.

FIG. 10 is a diagram illustrating a second example of a method of identifying a corresponding point group of a boundary of a predetermined site in a case where there is a side branch.

FIG. 11 is a diagram illustrating an example of a corrected 3-D image in a case where there is a side branch.

FIG. 12 is a diagram illustrating a first condition when medical image data is acquired.

FIG. 13 is a diagram illustrating a second condition when medical image data is acquired.

FIG. 14 is a diagram illustrating a third condition when medical image data is acquired.

FIG. 15 is a diagram illustrating an example of a correction method in the case of frame-out.

FIG. 16 is a diagram illustrating an example of a correction method in the case of an artifact.

FIG. 17 is a diagram illustrating a display example of a 3-D image of a blood vessel by the information processing device.

FIG. 18 is a diagram illustrating a procedure performed by the information processing device.

### DETAILED DESCRIPTION

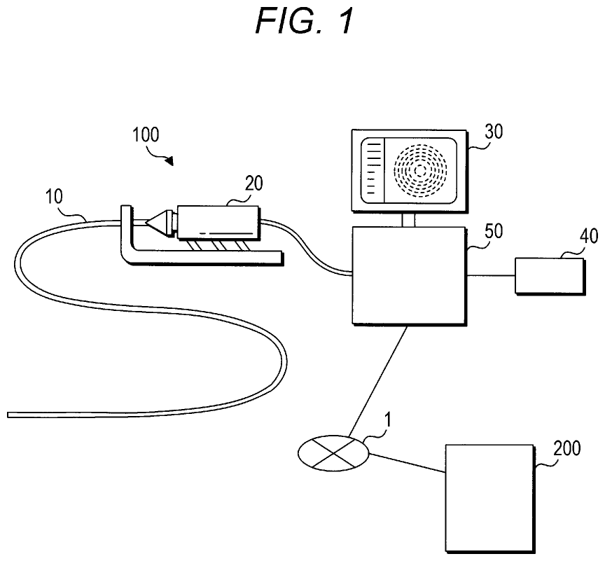

Hereinafter, embodiments of the present disclosure will be described. FIG. 1 is a diagram illustrating a configuration of an image diagnosis system 100 according to an embodiment. The image diagnosis system 100 is a device for performing intravascular imaging (image diagnosis) used for cardiac catheter treatment or PCI. The cardiac catheter treatment is a method of treating a narrowed portion of a coronary artery by inserting a catheter from a blood vessel such as a base of a leg, an arm, or a wrist. For intravascular imaging, there are two methods: an IVUS method; and an optical coherence tomography (e.g., OFDI or OCT) method. The IVUS uses reflection of an ultrasonic wave to generate a tomographic image of a blood vessel. Specifically, a thin catheter equipped with an ultra-small sensor at the distal end is inserted into the coronary artery, and after passing to a lesion, a medical image in the blood vessel can be generated by an ultrasonic wave transmitted from the sensor. The OFDI uses near-infrared rays to generate a high-resolution image of a blood vessel. Specifically, similarly to the IVUS, a catheter is inserted into a blood vessel, near-infrared rays are emitted from a distal end portion, a cross section of the blood vessel is measured by interferometry, and a medical image is generated. In addition, the OCT is intravascular image diagnosis to which near-infrared rays and optical fiber technology are applied. In the present specification, the medical image or medical image data includes one generated by the IVUS, the OFDI, or the OCT, but the case of using the IVUS method will be mainly described below.

The image diagnosis system 100 includes a catheter 10, a motor drive unit (MDU) 20, a display 30, an input device 40, and an information processing apparatus 50. A server 200 is connected to the information processing apparatus 50 via the communication network 1.

The catheter 10 is an image diagnosis catheter for obtaining an ultrasonic tomographic image of a luminal organ such as a blood vessel by the IVUS method. The catheter 10 has an ultrasonic probe at a distal end portion for obtaining an ultrasonic tomographic image of a blood vessel. The ultrasonic probe includes an ultrasound transducer that emits an ultrasonic wave in a blood vessel, and an ultrasonic sensor that receives a reflected wave (i.e., ultrasonic echo) reflected by a structure such as a biological tissue of the blood vessel or a medical device. The ultrasonic probe is movable back and forth by an operator in the longitudinal direction of the blood vessel while rotating in the circumferential direction of the blood vessel.

The MDU 20 is a drive device to which the catheter 10 can be detachably attached, and drives a built-in motor according to an operation of a medical worker to control behavior of the catheter 10 inserted into a blood vessel. The MDU 20 can be rotated in the circumferential direction while moving the ultrasonic probe of the catheter 10 from the distal end side to the proximal end side (i.e., pull-back operation). The ultrasonic probe continuously scans the inside of the blood vessel at predetermined time intervals, and outputs reflected wave data of the detected ultrasonic wave to the information processing apparatus 50.

The information processing apparatus 50 is a medical image processing apparatus that generates time-series (i.e., a plurality of frames of) medical image data including a tomographic image of a blood vessel on the basis of reflected wave data output from the ultrasonic probe of the catheter 10. Since the ultrasonic probe scans the inside of the blood vessel while moving from the distal end side to the proximal end side in the blood vessel, a plurality of medical images in chronological order is tomographic images of the blood vessel observed at a plurality of points from the distal end side to the proximal end side.

The display 30 includes a liquid crystal display (LCD) panel, an organic EL display panel, and the like, and can display a processing result by the information processing apparatus 50. Furthermore, the display 30 can display the medical image generated by the information processing apparatus 50.

The input device 40 includes a keyboard and a mouse that receives inputs of various setting values, operations of the information processing apparatus 50, and the like when a medical operation is performed. The input device 40 may be a touch panel, a software key, a hardware key, or the like provided in the display 30.

The server 200 is, for example, a data server, and may include an image database (DB) in which the medical image data is stored.

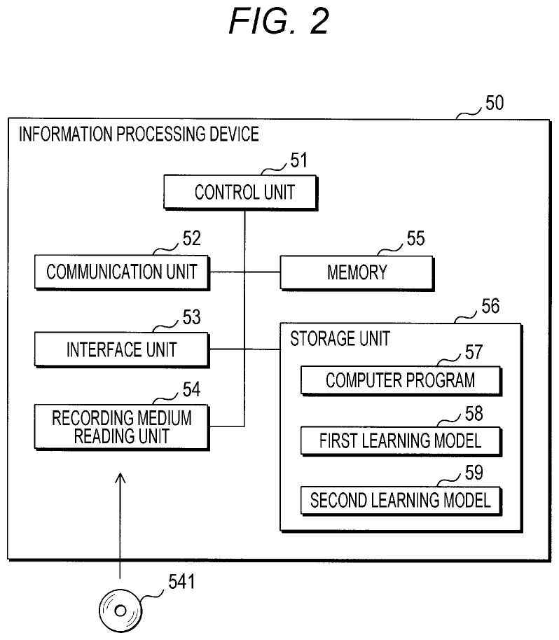

FIG. 2 is a diagram illustrating a configuration of the information processing apparatus 50. The information processing apparatus 50 includes a control unit 51 that controls the entire information processing apparatus 50, a communication unit 52, an interface unit 53, a recording medium reading unit 54, a memory 55, and a storage unit 56.

The control unit 51 is a controller or control circuit that includes at least one processor, e.g., a central processing unit (CPU), a micro-processing unit (MPU), a graphics processing unit (GPU), a general-purpose computing on graphics processing unit (GPGPU), and a tensor processing unit (TPU). Furthermore, the control unit 51 may be configured by combining a digital signal processor (DSP), a field-programmable gate array (FPGA) quantum processor, and the like. The control unit 51 performs the functions of a first acquisition unit, a second acquisition unit, an identification unit, and a generation unit according to a computer program 57 described later.

The memory 55 includes a static random access memory (SRAM), a dynamic random access memory (DRAM), or a flash memory.

The communication unit 52 is a network interface circuit that includes, for example, a communication module and has a communication function with the server 200 via the communication network 1. Furthermore, the communication unit 52 may have a communication function with an external device (not illustrated) connected to the communication network 1.

The interface unit 53 includes one or more interface circuits connectable to the catheter 10 or MDU 20, the display 30, and the input device 40. The information processing apparatus 50 can transmit and receive data and information to and from the catheter 10, the display 30, and the input device 40 via the interface unit 53.

The recording medium reading unit 54 can include, for example, an optical disk drive, and can read a computer program recorded on the recording medium 541 (for example, an optically readable disk storage medium such as a CD-ROM) by the recording medium reading unit 54 and store the computer program in the storage unit 56. The computer program 57 is loaded onto the memory 55 and executed by the control unit 51. Note that the computer program 57 may be downloaded from an external device via the communication unit 52 and stored in the storage unit 56.

The storage unit 56 is, for example, a hard disk drive (HDD), a semiconductor memory such as a solid state drive (SSD), or the like, and can store necessary information. The storage unit 56 can store a first learning model 58 and a second learning model 59 in addition to the computer program 57, which are described later. The first learning model 58 and the second learning model 59 include a model before training, a model in the middle of training, or a trained model.

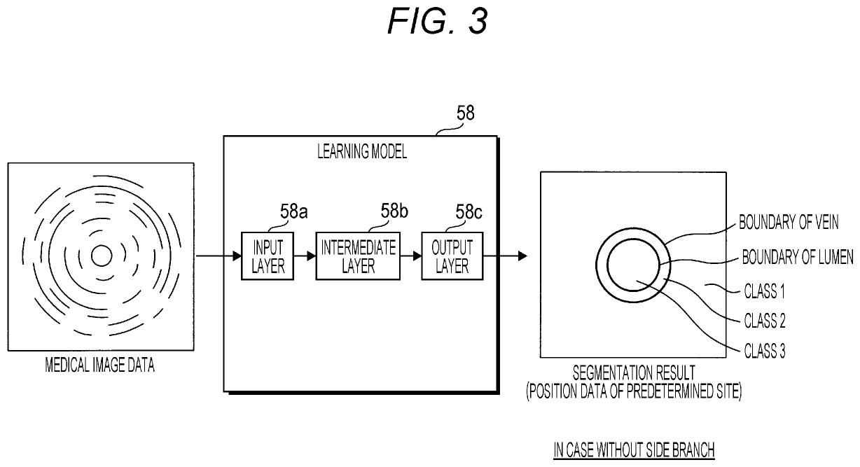

FIG. 3 is a diagram illustrating a first example of the configuration of the first learning model 58. The first learning model 58 is software executable on one or more processors and includes an input layer 58a, an intermediate layer 58b, and an output layer 58c, and is, for example, a convolutional neural network such as U-Net, a generative adversarial network (GAN), SegNet, or the like. The intermediate layer 58b includes a plurality of encoders and a plurality of decoders. A plurality of encoders performs convolution processing on medical image data input to the input layer 58a. Upsampling (i.e., deconvolution) processing is performed on the image convolved by the encoders by a plurality of decoders. When decoding the convoluted image, a process to add a feature map generated by the encoders to the image to be subjected to the deconvolution processing is performed. As a result, the position information lost by the convolution processing can be retained, and more accurate segmentation can be output.

The first learning model 58 is trained to output segmentation data when the medical image data is input. The segmentation data indicates a class of each pixel of the medical image data. The segmentation data may indicate, for example, three classes: classes 1, 2, and 3. Class 1 indicates background, that is a region outside the blood vessel. Class 2 indicates plaques and media, a region of the blood vessel containing plaques. Class 3 indicates a lumen of a blood vessel. Therefore, the first learning model 58 determines a boundary between a pixel classified into Class 2 and a pixel classified into Class 3 to be a boundary of the lumen, and a boundary between a pixel classified into Class 1 and a pixel classified into Class 2 to be a boundary of the blood vessel. That is, when the medical image data is input in the first learning model 58, the first learning model 58 can output position data indicating each of the boundary of the lumen and the boundary of the blood vessel. The position data is coordinate data of pixels indicating the boundary of the lumen and the boundary of the blood vessel.

A method for training the first learning model 58 can be as follows. First, first training data including medical image data indicating a cross-sectional image of a blood vessel and segmentation data indicating a class of each pixel of the medical image data is acquired. For example, the information may be collected and stored in the server 200 and acquired from the server 200. Next, the first learning model 58 is trained on the basis of the first training data to output segmentation data when the medical image data indicating a cross-sectional image of a blood vessel is input to the first learning model 58. In other words, on the basis of the first training data, the first learning model 58 is trained so as to output the position data of each of the boundary of the lumen and the boundary of the blood vessel when the medical image data indicating the cross-sectional image of the blood vessel is input to the first learning model 58.

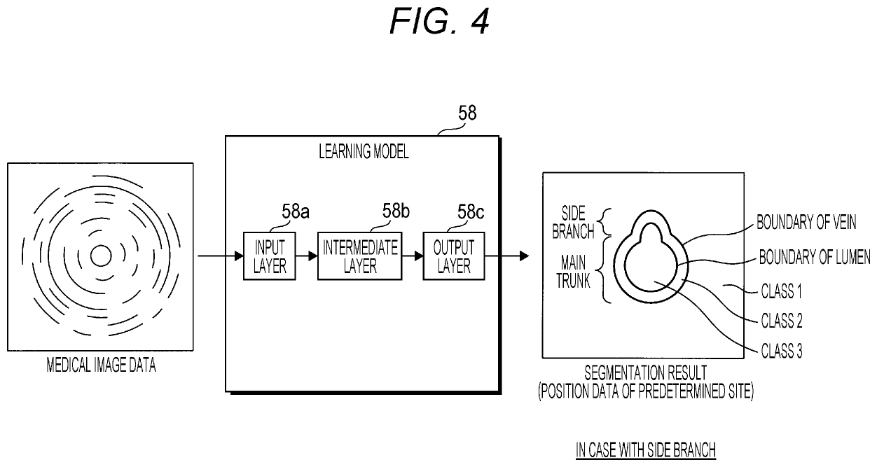

FIG. 4 is a diagram illustrating a second example of the configuration of the first learning model 58. The configuration of the first learning model 58 of the second example is similar to that of the first example, but the difference from the first example is that not only in a case where a side branch does not exist in a blood vessel but also in a case where a side branch exists in a blood vessel, position data indicating each of the boundary of the lumen and the boundary of the blood vessel can be output when medical image data is input, as in the case of the first example. The first learning model 58 of the second example can output the position data of the boundary of the lumen and the boundary of the blood vessel of each of the main trunk of the blood vessel and the side branch connected to the main trunk.

A method for training the first learning model 58 of the second example can be as follows. First, medical image data indicating a cross-sectional image of a blood vessel having a side branch, and first training data including position data of each of a boundary of a lumen of the blood vessel and a boundary of the blood vessel are acquired. For example, the first training data may be collected and stored in the server 200 and acquired from the server 200. Next, on the basis of the first training data, the first learning model 58 of the second example is trained so as to output the position data of each of the boundary of the lumen of the blood vessel with the side branch and the boundary of the blood vessel when the medical image data indicating the cross-sectional image of the blood vessel is input to the first learning model 58. Note that the first training data can include both medical image data in which there is the side branch in the cross-sectional image of the blood vessel and medical image data in which the side branch is not present in the cross-sectional image of the blood vessel. In the present embodiment, the first learning model 58 of the second example can be used.

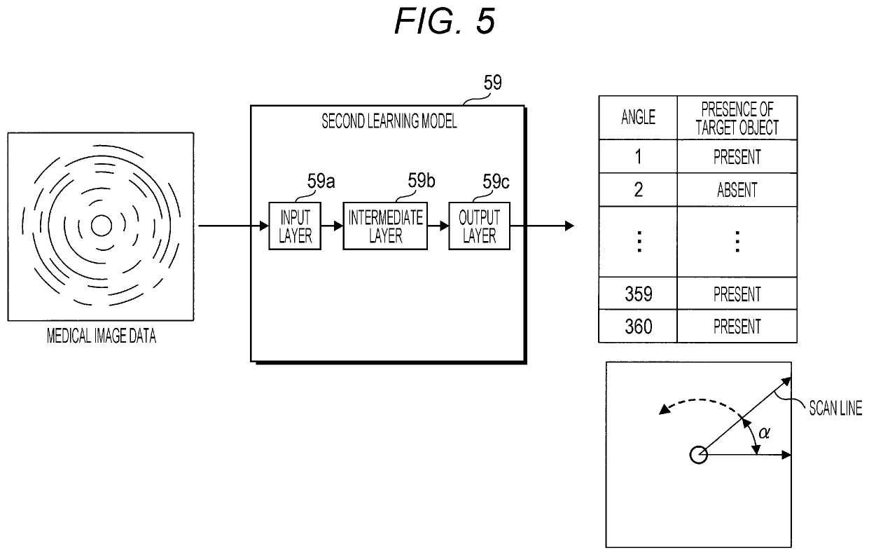

FIG. 5 is a diagram illustrating an example of a configuration of the second learning model 59. The second learning model 59 is trained by a medical image such as an IVUS image and object data indicating whether or not an object exists on a scan line of the image at each angle (e.g., the table shown in FIG. 5). The second learning model 59 includes an input layer 59a, an intermediate layer 59b, and an output layer 59c, and is, for example, a convolutional neural network. The intermediate layer 59b includes a plurality of convolution layers, a plurality of pooling layers, and a fully connected layer. The second learning model 59 is trained to output the presence or absence of the target object when the medical image data is input. The medical image data input to the input layer 59a is subjected to a convolution operation by a convolution filter (also referred to as a filter) in a convolution layer, and a feature map is output. The pooling layer performs processing of reducing the size of the feature map output from the convolution layer. With the pooling layer, for example, even if a feature portion is slightly deformed or displaced in the medical image, a difference due to the deformation or displacement can be absorbed to extract the feature portion.

The output layer 59c includes 360 nodes, and outputs a value (for example, with object: 1, without object: 0, etc.) according to the presence or absence of the target object and a type of the target object on a scanning line in a radial direction with a predetermined position of the medical image as a center. The scanning line is composed of 360 line segments obtained by dividing the entire circumference into 360 equal parts.

Embodiments of the present disclosure are not limited to the configuration in which the entire circumference is divided into 360 equal parts, and for example, the entire circumference may be equally divided into an appropriate number, such as two equal parts, three equal parts, or 36 equal parts. When there is the target object on the medical image, a value indicating the presence of the target object is output over a plurality of scanning lines. The second learning model 59 may be trained to detect the presence or absence of the target object in the medical image without using the operation line.

The target object includes a lesion and a structure. The lesion includes, for example, disassociation, a protrusion, or a thrombus that occurs only in the superficial layers of the lumen. The lesion also includes calcified or attenuating plaques that develop from the superficial layer of the lumen to the blood vessel. The structure includes a stent or a guidewire.

As described above, in a case where the medical image data indicating the cross-sectional image of the blood vessel is input, the computer program 57 can input the acquired medical image data to the second learning model 59 that outputs the presence or absence of the target object of the blood vessel and output the presence or absence of the target object.

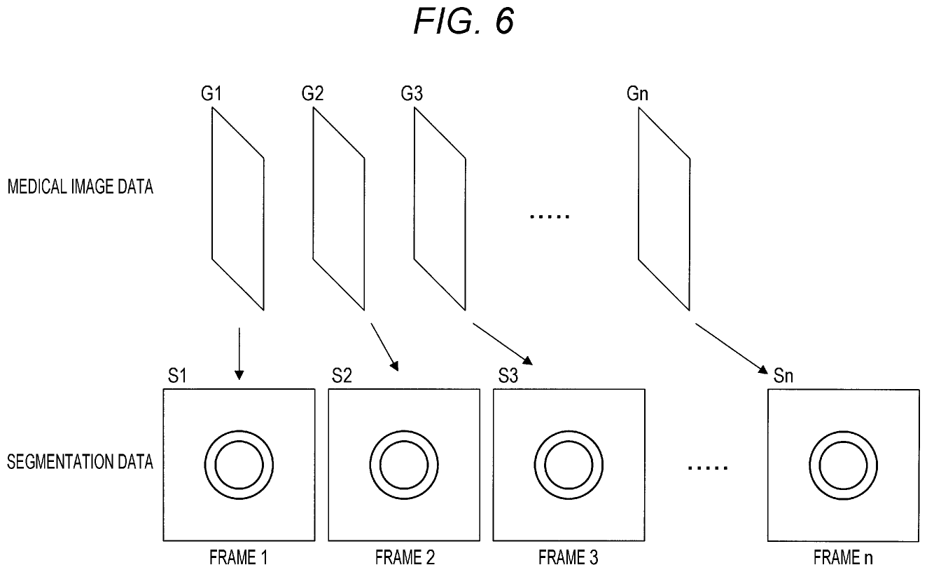

FIG. 6 is a diagram illustrating an example of segmentation data output by the first learning model 58. A plurality of pieces of medical image data (G1, G2, G3, . . . , Gn) corresponding to cross-sectional images of a plurality of frames (frames 1 to n) are acquired from a plurality of time-series cross-sectional images of the blood vessel obtained by one pull-back operation. The acquired medical image data may be all or a part of the cross-sectional image obtained by one pull-back operation. The acquired medical image data is input data to the first learning model 58. Conditions for acquiring the medical image data will be described later. The first learning model 58 outputs segmentation data (S1, S2, S3, . . . , Sn) respectively corresponding to the frames 1 to n.

As described in FIG. 4, each segmentation data includes the position data of the main trunk of the blood vessel and the boundary of the lumen and the boundary of the blood vessel of each side branch (when present) connected to the main trunk.

Although not illustrated, the acquired medical image data (G1, G2, G3, . . . , Gn) is input data to the second learning model 59. The second learning model 59 outputs target object data indicating the presence or absence of a target object corresponding to each of the frames 1 to n. As described with reference to FIG. 5, the target object data indicates the presence or absence of the target object on the 360 scanning lines. When there is the target object, the value of presence of the target object is output over a plurality of scanning lines, so that it is possible to locate the position of presence of the target object on the medical image to some extent.

Next, a method for generating a 3-D image of a blood vessel will be described.

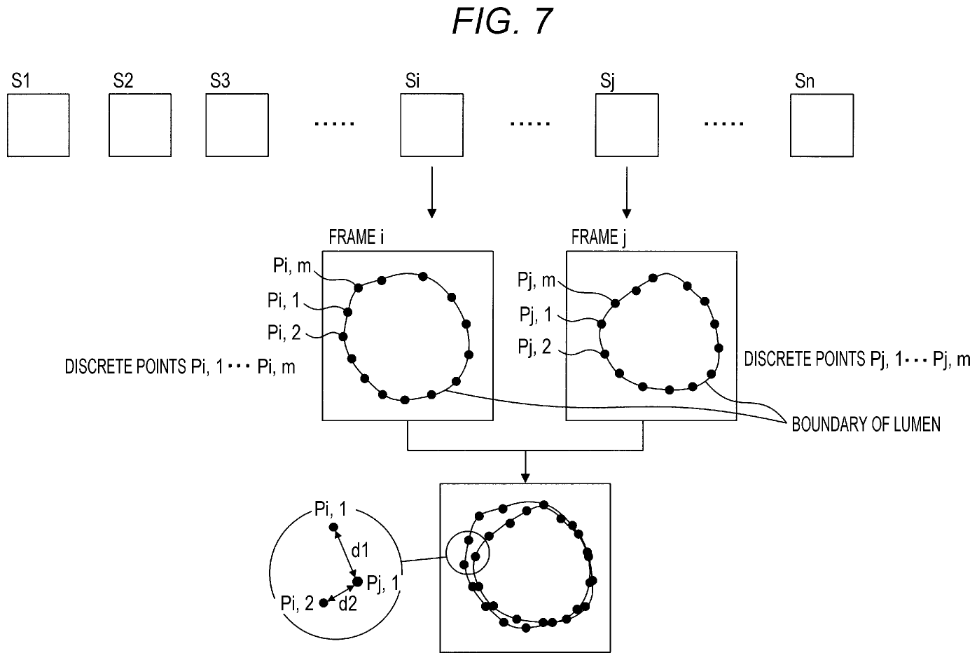

FIG. 7 is a diagram illustrating a method of identifying a corresponding point group of a boundary of a predetermined site in a case where there is no side branch. Hereinafter, the boundary of the lumen will be described as the predetermined site, but the predetermined site also includes the boundary of the blood vessel. The segmentation data output by the first learning model 58 is assumed as S1, S2, S3, . . . , Si, . . . , Sj, . . . , and Sn, where n is the number of frames, j is the number of the frame of interest, and i is the number of the frame corresponding to the frame of interest j. The frame of interest j and the corresponding frame i are necessary frames for identifying the corresponding point group. Note that the frame of interest j and the corresponding frame i may not be adjacent frames, and another frame may exist between the frames of interest j and the corresponding frame i.

A discrete point on the boundary of the lumen indicated by the segmentation data Si of the frame i is represented by P (i, m), and a discrete point on the boundary of the lumen indicated by the segmentation data Sj of the frame j is represented by P (j, m), m being a number from 1 to m, indicating the number of discrete points. Examples of a method for identifying the discrete points include: (1) a method of sequentially identifying the discrete points at the same angle along the boundary; (2) a method of identifying the discrete points such that the distance between the discrete points is constant; and (3) a method of identifying the discrete points such that the number of the discrete points is constant. As a result, regardless of the shape of the boundary, it is possible to acquire discrete points in a well-balanced manner while suppressing excessive torsion and the like. The number of the discrete point on the boundary of the blood vessel may be less than or equal to the number of the discrete point on the boundary of the lumen. As a result, the number of discrete points on the boundary of the blood vessel can be reduced, and the visibility of the mesh of the lumen can be improved.

A distance between the discrete point P (j, 1) of the segmentation data Sj of the frame j and each of the m discrete points P (i, m) (m=1 to m) of the segmentation data Si of the frame i is calculated, and the discrete point P (i, m) having the shortest distance is identified as a corresponding point. In the example of FIG. 7, when the distances d1 and d2 between the discrete point P (j, 1) and the discrete points P (i, 1) and P (i, 2) are calculated and the calculated distances d1 and d2 are compared, the distance d2 is shorter than the distance d1, and thus the discrete point P (i, 2) is selected. A similar comparison is made for other discrete points.

By performing similar processing on each discrete point P (j, m) of the segmentation data Sj of the frame j, it is possible to identify the corresponding point group of the boundary of the predetermined site between the frame i and the frame j. In addition, since the cross-sectional shape of the blood vessel can be obtained from the segmentation data of each frame, the gravity center of the blood vessel can also be obtained. For example, the gravity center may be the center of the average lumen diameter. The average lumen diameter D can be obtained by D=2 ×√(S/π) using an area S of the region occupied by the pixel indicating the lumen.

As described above, the computer program 57 can identify the discrete point group of the boundary on the basis of the acquired segmentation data, and identify the corresponding point group of the boundary by associating the discrete point groups with each other in two different frames (for example, frame j and frame i) selected from a plurality of frames. More specifically, the computer program 57 can calculate the distance between the discrete point groups in two different frames selected from the plurality of frames, and identify the corresponding point group of the boundary by associating the discrete point groups having the smallest calculated distance with each other.

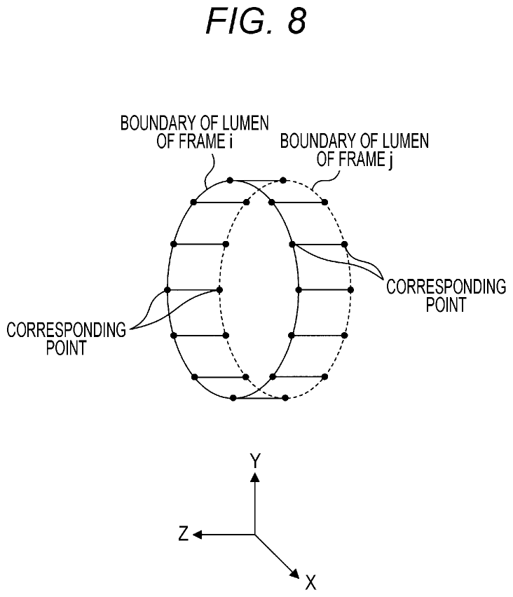

FIG. 8 is a diagram illustrating an example of a method of connecting corresponding point groups. It is assumed that a discrete point group on the lumen boundary indicated by the segmentation data Si of the frame i and a discrete point group on the lumen boundary indicated by the segmentation data Sj of the frame j are associated with each other. The discrete points associated with each other are referred to as corresponding points, and the corresponding points on the boundary are collectively referred to as a corresponding point group. The segmentation data Si of the frame i and the segmentation data Sj of the frame j are separated at an appropriate interval from each other and arranged along the Z-axis direction. The Z axis indicates the long axis direction of the blood vessel. The corresponding points (i.e., corresponding point group) of the frames i and j are connected by a straight line in the Z-axis direction. In addition, corresponding points adjacent on the boundary are connected by a straight line along the boundary. As a result, it is possible to generate a 3-D image in which the lumen boundaries indicated by the respective segmentation data of the frames i and j are connected in a mesh shape. Similarly, a 3-D mesh image can be generated for the blood vessel boundary. By performing similar processing on all the frames 1 to n, a 3-D mesh image of the blood vessel can be generated.

As described above, the computer program 57 can generate a 3-D mesh image of a blood vessel by connecting corresponding point groups identified in two different frames selected from a plurality of frames over a plurality of frames and connecting corresponding point groups identified in each frame along a boundary.

When the side branch is present, if a 3-D image of the blood vessel is generated by the method illustrated in FIG. 7, an open cross section of the side branch cannot be illustrated on the 3-D image. Therefore, when there is a side branch, it is necessary to correct the method of identifying the corresponding point group using the following method. Hereinafter, a correction method will be described.

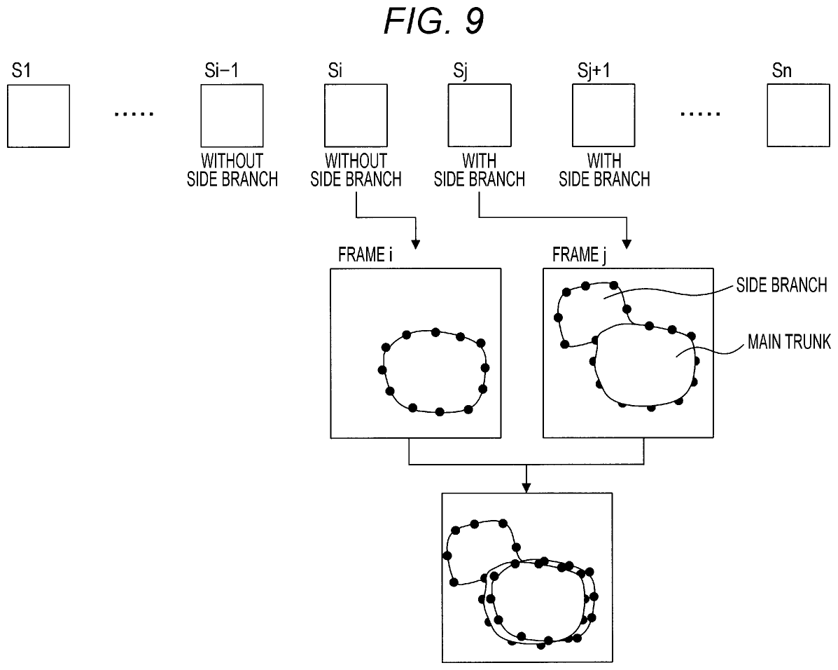

FIG. 9 is a diagram illustrating a first example of a method of identifying a corresponding point group of a boundary of a predetermined site in a case where there is a side branch. The presence or absence of a side branch can be detected by using the first learning model 58 illustrated in FIG. 4. The presence or absence of the side branch can be determined by the eccentricity of the blood vessel cross-sectional shape. For example, the eccentricity can be obtained by calculating the maximum diameter D1 and the minimum diameter D2 of the lumen diameter on the basis of the boundary of the lumen. The eccentricity can be calculated by an equation of eccentricity=(maximum diameter D1−minimum diameter D2)/maximum diameter D1. The presence or absence of a side branch can be determined according to whether or not the eccentricity is greater than or equal to a predetermined threshold. Instead of the eccentricity, the circularity may be calculated. The circularity is the ratio of the area of the inner region of the blood vessel boundary to the circumferential length of the blood vessel boundary. It can be determined that the closer the circularity is to the ratio between the area of the circle and the length of the circumference, the higher the circularity is and the lower the possibility that a side branch is shown. In addition, as another determination example of the presence or absence of a side branch, a value obtained by comparing the diameter (i.e., the maximum diameter and minimum diameter) of the target blood vessel boundary with the diameter of the blood vessel boundary with respect to the scanned tomographic image is calculated as a parameter, and the presence or absence of a side branch can be determined according to whether or not the diameter suddenly changes by a predetermined ratio or more and by a predetermined length or more. As still another determination example, determination may be made using a learning model for determination trained to output accuracy corresponding to the possibility that a side branch is shown in a case where data of a lumen boundary and a blood vessel boundary is input.

As illustrated in FIG. 9, it is assumed that there is no side branch in the segmentation data S1, . . . , S (i−1), and Si, and there is a side branch in the segmentation data Sj, S (j+1). In this case, as the corresponding frame, a frame without a side branch and a frame with a side branch can be associated. In the example of FIG. 9, the segmentation data Si of the frame i is associated with the segmentation data Sj of the frame j. The discrete point group of the lumen boundary of the segmentation Si and the discrete point group of the lumen boundary of the main trunk of the segmentation Sj are associated with each other and identified as a corresponding point group, and subjected to the same processing as the processing illustrated in FIG. 7. On the other hand, the discrete point group of the lumen boundary of the side branch of the segmentation Sj is left as is, and not subjected to the connection processing. The gravity center of the blood vessel in which the side branch is present can be corrected using the region of only the main trunk.

As described above, the computer program 57 can determine the presence or absence of a side branch of a blood vessel on the basis of the acquired segmentation data, and when there is a side branch, the side branch can be associated with a frame with no side branch, and the computer program can be configured not to connect a discrete point group corresponding to the side branch among the discrete point groups identified in the frame with the side branch. As a result, it is possible to prevent a situation in which the entire side branch is connected in a mesh shape and the opening cross section of the side branch does not appear on the 3-D image, and it is possible to clearly and intuitively locate the position of the side branch on the 3-D image.

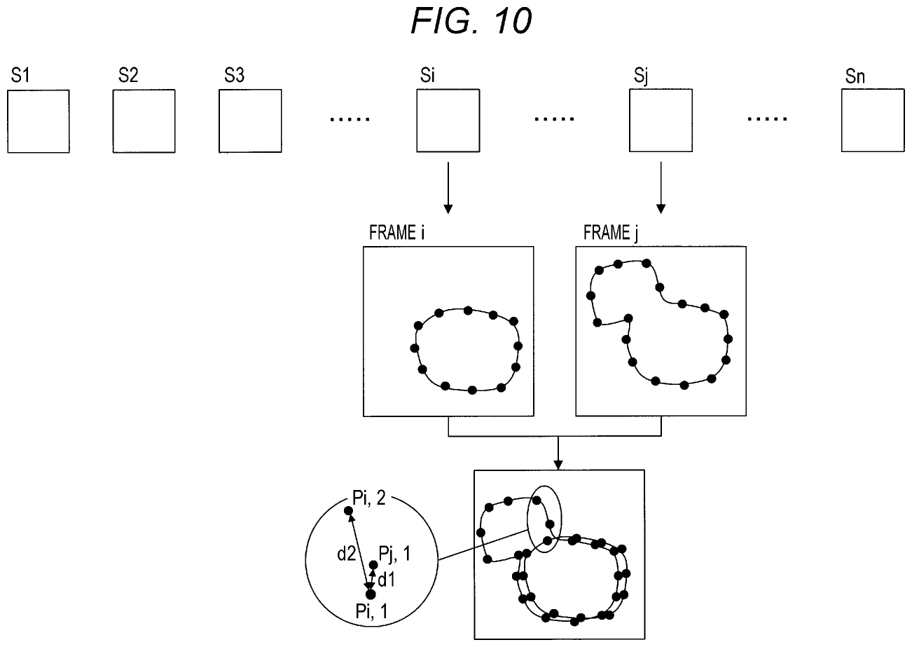

FIG. 10 is a diagram illustrating a second example of a method of identifying a corresponding point group of a boundary of a predetermined site in a case where there is a side branch. In the example of FIG. 10, it is not necessary to detect a side branch by the first learning model 58. As illustrated in FIG. 10, distances d1 and d2 between the discrete point P (j, 1) and the discrete points P (i, 1) and P (i, 2), respectively, are calculated. Whether to perform association is determined according to whether the calculated distances d1 and d2 are greater than or equal to a predetermined threshold. For example, since the distance d1 is smaller than the threshold, association is performed. On the other hand, since the distance d2 is longer than the threshold, no association is performed. As described above, when the corresponding points are identified, if the distance between the discrete points is greater than or equal to the threshold, the discrete points are not connected. A similar comparison is made for other discrete points.

As described above, the computer program 57 can identify the discrete point group of the boundary on the basis of the acquired segmentation data, calculate the distance between the discrete point groups in two different frames selected from a plurality of frames, and do not associate the discrete point groups with each other in a case where the calculated distance is greater than or equal to a predetermined threshold value. As a result, it is possible to prevent a situation in which the entire side branch is connected in a mesh shape and the opening cross section of the side branch does not appear on the 3-D image, and it is possible to clearly and intuitively locate the position of the side branch on the 3-D image.

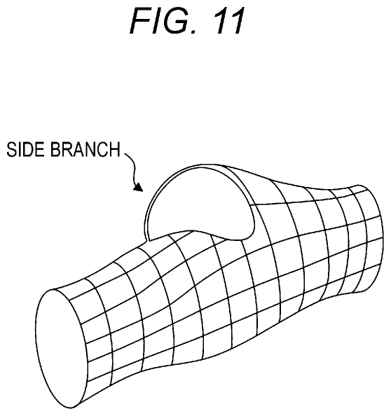

FIG. 11 is a diagram illustrating an example of a corrected 3-D image in a case where there is a side branch, which is an anatomical feature. As illustrated in FIGS. 9 and 10, by not connecting the discrete point group of the lumen boundary and the blood vessel boundary of the side branch to the discrete point group of the boundary of the segmentation data of another frame, it is possible to prevent a situation in which the entire side branch is connected in a mesh shape and the opening cross section of the side branch does not appear on the 3-D image, and it is possible to clearly and intuitively locate the position of the side branch or anatomical feature on the 3-D image.

Next, conditions for acquiring medical image data (G1, G2, G3, . . . , Gn) including cross-sectional images of a plurality of frames (frames 1 to n) will be described.

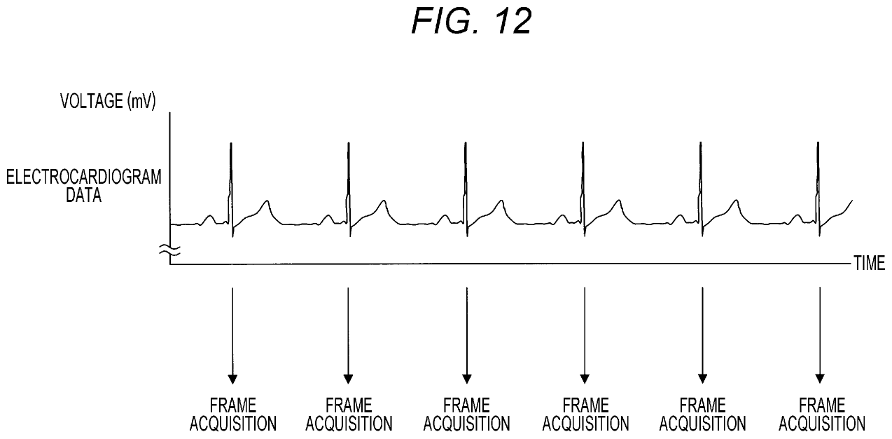

FIG. 12 is a diagram illustrating a first condition for acquiring the medical image data. As illustrated in FIG. 12, a frame may be acquired in synchronization with a specific cardiac cycle (in the example of the diagram, the timing of the peak waveform) of the electrocardiogram data. The frame acquired in synchronization with the specific cardiac cycle (i.e., the peak waveform) may be one frame or a plurality of frames. As a result, it is possible to generate a more accurate 3-D image by suppressing adverse effects such as distortion generated in the 3-D image due to expansion and contraction of the blood vessel.

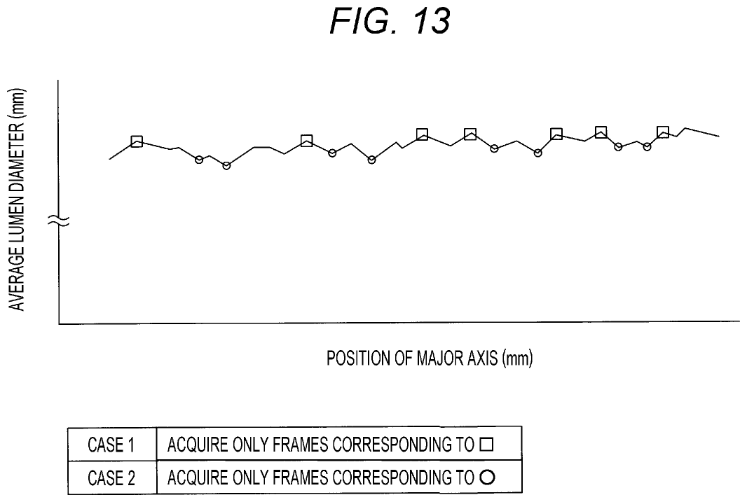

FIG. 13 is a diagram illustrating a second condition for acquiring the medical image data. In FIG. 13, the average lumen diameter of the blood vessel calculated by the segmentation processing by the first learning model 58 is plotted along the long axis direction with respect to the medical image data obtained by one pull-back operation. When medical image data of a plurality of frames is acquired, only specific medical image data can be selected and acquired by autocorrelation or the like. For example, only the frames having the greatest average lumen diameter as indicated by squares may be acquired. Alternatively, only the frames having the smallest average lumen diameter as indicated by circles may be acquired. As a result, it is possible to generate a more accurate 3-D image by suppressing adverse effects such as distortion generated in the 3-D image due to expansion and contraction of the blood vessel.

Note that, although not illustrated, a frame corresponding to a portion where the timing at which the average lumen diameter is the greatest and the timing of the peak waveform of the electrocardiogram data do not match in the middle of the transition of the average lumen diameter as illustrated in FIG. 13 may include some factors of erroneous determination, and thus it is better not to use the frame for the segmentation processing.

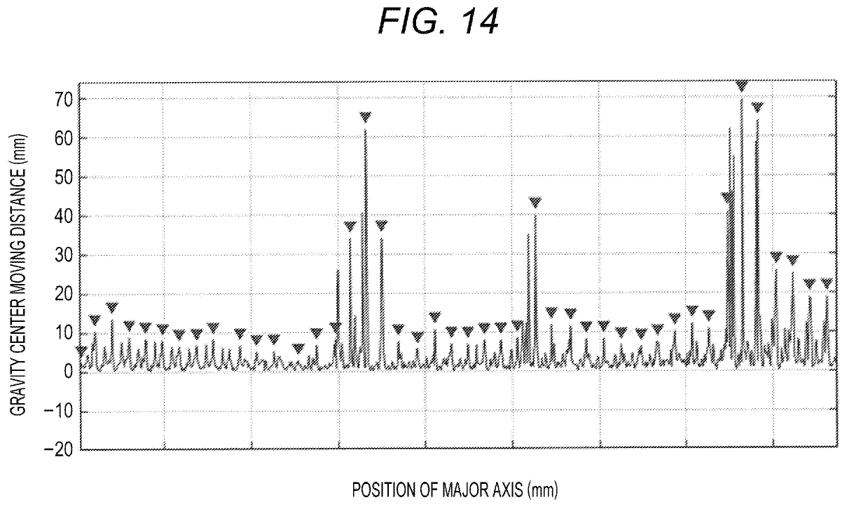

FIG. 14 is a diagram illustrating a third condition for acquiring the medical image data. In FIG. 14, the gravity center moving distance of the blood vessel calculated by the segmentation processing by the first learning model 58 is plotted along the long axis direction with respect to the medical image data obtained by one pull-back operation. The gravity center moving distance is calculated for each frame, and can be expressed as a distance between the gravity center position on the cross-sectional image in the frame of interest and the gravity center position on the cross-sectional image in the frame immediately before the frame of interest. When medical image data of a plurality of frames is acquired, it is possible to select and acquire only specific medical image data by detecting an extreme value of the gravity center moving distance plotted in the long axis direction. For example, only the frames having the greatest gravity center moving distance as indicated by inverted triangles in FIG. 14 may be acquired. As a result, it is possible to generate a more accurate 3-D image by suppressing adverse effects such as distortion generated in the 3-D image due to expansion and contraction of the blood vessel.

As described above, the computer program 57 can acquire medical image data indicating cross-sectional images of the plurality of frames on the basis of a gravity center moving distance of the blood vessel, predetermined cardiac cycle data, or correlation data of a predetermined index of the predetermined site.

Next, a correction method in a case where a lumen boundary of a blood vessel or a blood vessel boundary is erroneously detected due to a frame-out exceeding a depth that can be acquired by the IVUS will be described.

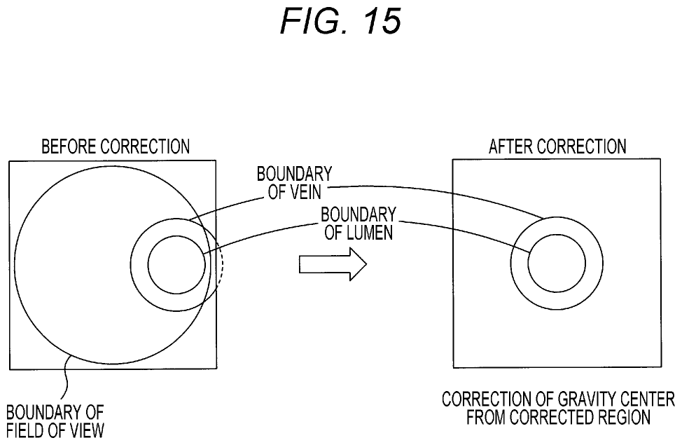

FIG. 15 is a diagram illustrating an example of a correction method in the case of frame-out. In FIG. 15, the diagram before correction illustrates a state in which a part of the blood vessel boundary exists outside the visual field boundary due to frame-out. In this case, on the basis of the position data (i.e., coordinates) of the blood vessel boundary existing in the visual field boundary, the blood vessel boundary outside the visual field boundary is interpolated using, for example, a spline or a circular fitting. The diagram after the correction illustrates the blood vessel boundary after the interpolation. Note that, in the example of FIG. 15, interpolation processing is not performed because the lumen boundary is not framed out, but interpolation can be similarly performed in a case where the lumen boundary is framed out. After interpolating the vessel boundary, the gravity center may be corrected from the blood vessel region. In addition, when the lumen boundary is interpolated, the gravity center may be corrected from the lumen region.

As described above, when the boundary of the predetermined site is outside of a visual field boundary, the computer program 57 can interpolate the boundary of the predetermined site on the basis of information of a boundary in the visual field boundary. As a result, even when the boundary of the lumen or the blood vessel exceeds the depth that can be acquired by the IVUS, the boundary of the lumen or the blood vessel can be accurately detected.

Next, a correction method in a case where the reliability of the boundary of the lumen or the blood vessel due to the segmentation decreases under the influence of an artifact will be described. The artifact includes noise and a structure such as a stent or a guidewire.

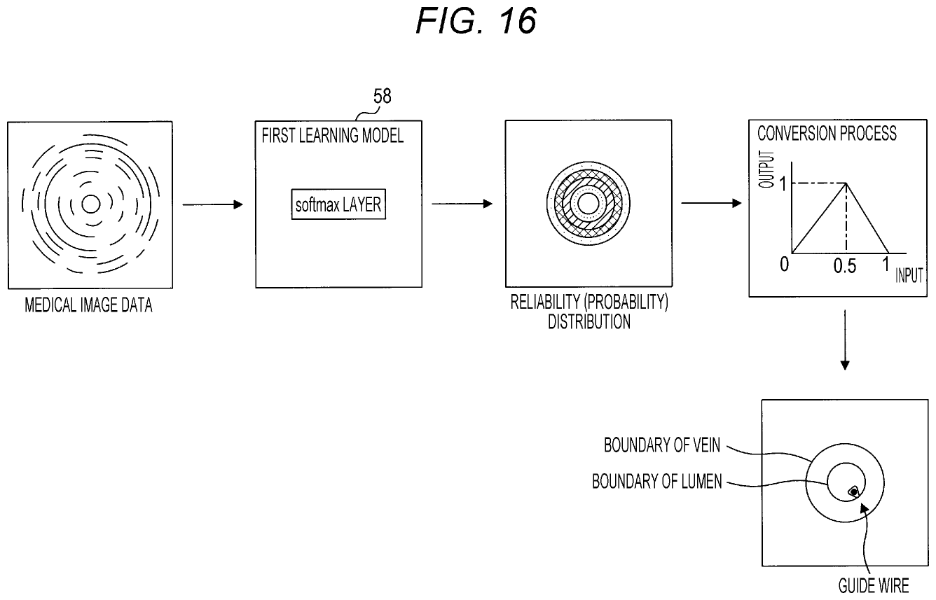

FIG. 16 is a diagram illustrating an example of a correction method in the case of an artifact. Note that FIG. 16 illustrates an example in the case of two classes, but the number of classes is not limited to two. The softmax layer of the first learning model 58 converts the segmentation result into a probability, and outputs the probability of the class of each pixel of the segmentation data as a reliability distribution. Specifically, for example, lumen likelihood (or blood vessel likelihood) is output as a numerical value from “0” to “1” for each pixel. For example, “1” indicates a high probability of being a lumen, and “0” indicates a high probability of being of a class other than the lumen. In a pixel in which outputs of two or more classes are similar, a prediction result of the segmentation is equivalent, which means that this is a portion that cannot be uniquely classified. By calculating the similarity of each class with respect to the reliability distribution, it is possible to obtain a result in which a region in which the prediction results of the segmentation are equivalent and cannot be uniquely classified is emphasized. In the example of FIG. 16, a guide wire is detected and shown in a predetermined color (e.g., a different color from the other part of the image). In this way, due to the influence of the artifact, it is possible to visualize pixels in which prediction results of segmentation are equivalent and which cannot be uniquely classified.

As described above, the computer program 57 can detect an artifact on the basis of reliability of segmentation data output by the first learning model 58. As a result, it is possible to visualize pixels that cannot be uniquely classified when the probability of class classification is around 0.5 due to the influence of the artifact.

The artifact detection processing is performed by the second learning model 59 illustrated in FIG. 5, and the portion of the artifact such as the guide wire, the stent, and the noise can be visualized by displaying the portion of the artifact in a display mode (for example, the color and pattern are changed) different from the portion of the blood vessel.

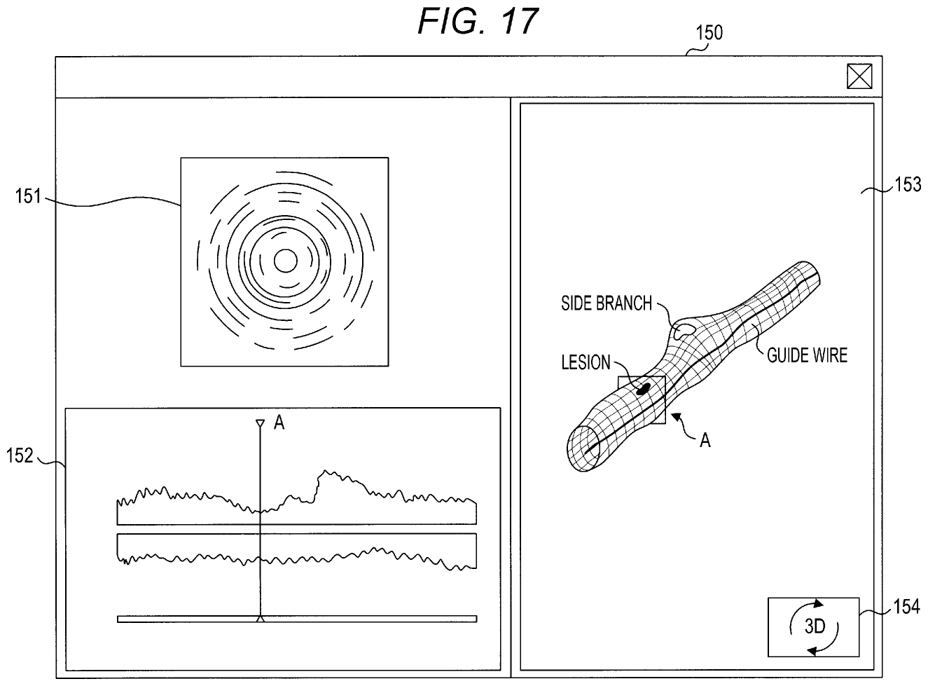

FIG. 17 is a diagram illustrating a display example of a 3-D image of a blood vessel by the information processing apparatus 50. The 3-D image display screen 150 includes a screen 151 for displaying a cross-sectional image of the blood vessel, a screen 152 for displaying a cross-sectional image along the long axis direction of the blood vessel, and a screen 153 for displaying a 3-D image of the blood vessel. In a case where a 3-D image of a blood vessel or a screen image along the long axis direction is displayed, distortion of the 3-D image or the cross-sectional image can be suppressed by aligning the gravity center calculated on the basis of the cross-sectional shape of the blood vessel obtained as a result of the segmentation processing of each frame along the long axis direction. In addition, instead of the gravity center in each frame, a 3-D image or a screen image along the long axis direction can be aligned on the basis of a structure having obvious continuity along the long axis direction of a guide wire or the like.

By sliding the marker indicated by the reference sign A on the screen 152 left and right, a cross-sectional image of the blood vessel corresponding to the position of the marker is displayed on the screen 151, and the position of the marker is displayed on the screen 153, so that the position on the 3-D image can be easily located.

In the 3-D image on the screen 153, for example, the blood vessel may be displayed as a 3-D mesh image. In addition, the lesion or the guide wire may be displayed in a display mode different from that of the blood vessel. For example, the lesion and the guide wire may be displayed in different colors or patterns. In addition, the lesion or the guide wire may be displayed as a volume, or may be displayed with different degrees of transparency. In addition, since the mesh is not connected to the portion where the side branch is present, the position of the side branch can be intuitively located. By operating the 3-D icon 154, the 3-D image of the blood vessel can be rotated by 360° to view the 3-D image from a desired direction. In the 3-D image on the screen 153, a rectangular frame is displayed.

As described above, the computer program 57 can display a 3-D mesh image of a blood vessel and display an artifact or a lesion in different display modes on the 3-D image.

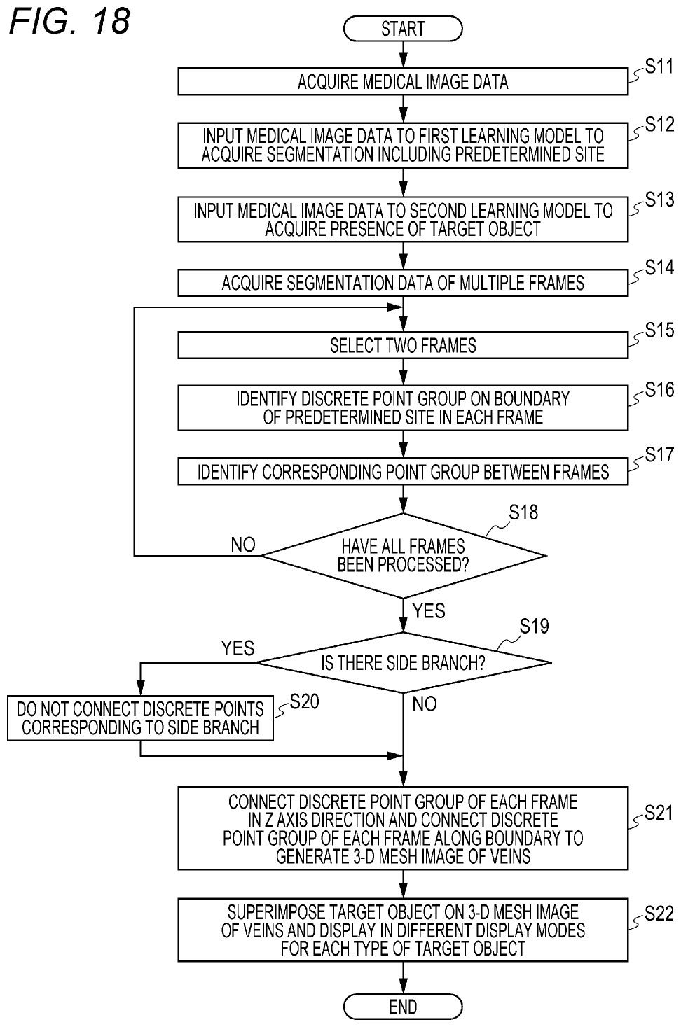

FIG. 18 is a diagram illustrating a procedure performed by the information processing apparatus 50. Hereinafter, for convenience, the control unit 51 will be described as the subject of the processing. The control unit 51 acquires medical image data (S11), inputs the acquired medical image data to the first learning model 58 to acquire segmentation data including a predetermined site (S12). The control unit 51 inputs the acquired medical image data to the second learning model 59 to acquire the presence or absence of the target object (S13). The target object includes a lesion or structure.

The control unit 51 acquires segmentation data of a plurality of frames (S14). For acquiring the plurality of frames, for example, the method illustrated in FIG. 12 or 13 can be used. The control unit 51 selects two frames (S15), identifies a discrete point group of a boundary of a predetermined site in each frame (S16), and identifies a corresponding point group of frames (S17). As a method for identifying the corresponding point group, for example, the method illustrated in FIG. 7 can be used.

The control unit 51 determines whether the processing of all the frames is completed (S18), and if the processing is not completed (NO in S18), continues the processing of S15. When the processing of all the frames is completed (YES in S18), the control unit 51 determines the presence or absence of a side branch (S19). For the determination of the presence or absence of a side branch, the method illustrated in FIG. 9 or 10 can be used.

When there is a side branch (YES in S19), the control unit 51 does not connect the discrete points corresponding to the side branch (S20), and performs the processing of S21 described later. When there is no side branch (NO in S19), the control unit 51 connects the corresponding point group of each frame in the Z-axis direction and connects the corresponding point group of each frame along the respective boundaries of the lumen and the blood vessel to generate a 3-D mesh image of the blood vessel (S21). The control unit 51 superimposes a target object (for example, a lesion, a structure, or the like) on the 3-D mesh image of the blood vessel, displays the target object in different display modes for each type of the target object (S22), and ends the processing. When the side branch is present in the blood vessel, as illustrated in FIG. 17, the 3-D image can be displayed in such a manner that the position of the side branch can be intuitively located.

As described above, the computer program 57 can acquire medical image data indicating cross-sectional images of a plurality of frames of a blood vessel, acquire segmentation data including a predetermined site for each frame by inputting the acquired medical image data to a first learning model 58 that outputs segmentation data including the predetermined site of the blood vessel in a case where medical image data indicating a cross-sectional image of a blood vessel is input, identify a corresponding point group of a boundary of the predetermined site in two different frames selected from the plurality of frames on the basis of the acquired segmentation data, and generate a 3-D image of the blood vessel on the basis of the identified corresponding point group.

By using the IVUS method, the plaque volume can also be determined.

In the above-described embodiments, the information processing apparatus 50 is configured to generate a 3-D image of a blood vessel, but embodiments of the present disclosure are not limited thereto. For example, the information processing apparatus 50 may be a client device, and the 3-D image generation processing may be performed by an external server, and the 3-D image may be acquired from the server.

What is claimed is:

1. A medical image processing apparatus for processing medical images of a luminal organ, comprising:
a first interface circuit connectable to a catheter having an ultrasonic probe and insertable into the luminal organ;
a second interface circuit connectable to a display; and
a processor configured to:
control the catheter to acquire a plurality of cross-sectional images of the luminal organ when the catheter is inserted into the luminal organ and moved along a longitudinal direction thereof,
input the acquired images into a first machine learning model that has been trained to classify each pixel in an image of a luminal organ and for each of the acquired images, obtain position data indicating a boundary between predetermined regions of the luminal organ based on segmentation data output from the first machine learning model that classifies each pixel of the acquired image,
select two of the images that are consecutive and identify a group of points corresponding to the boundary in each of the selected images based on the position data,
associate one or more of the points in one of the selected images with one or more of the points in the other image,
generate a 3-D image of the luminal organ in which said one or more of the points in one of the selected images are respectively connected to the associated points in the other image, and
control the display to display the generated 3-D image.

2. The medical image processing apparatus according to claim 1, wherein the processor is configured to:
determine a difference between each of the points in one of the selected images and each of the points in the other image, and
associate one of the points in one of the selected images and one of the points in the other image, a difference of which is smallest.

3. The medical image processing apparatus according to claim 2, wherein the processor is configured to determine not to associate one of the points in one of the selected images and one of the points in the other image, a difference of which is greater than or equal to a threshold.

4. The medical image processing apparatus according to claim 1, wherein in the 3-D image, said one or more of the points in one of the selected images are connected, and said one or more of the points in the other image are connected.

5. The medical image processing apparatus according to claim 1, wherein the processor is configured to:
determine whether a side branch of the luminal organ is shown in each of the images based on the segmentation data, and
determine not to connect one or more of the points in one of the selected images corresponding to the side branch and the associated points in the other image.

6. The medical image processing apparatus according to claim 1, wherein the processor is configured to:
determine whether a part of the boundary falls outside of each of the images based on the segmentation data, and
upon determining that a part of the boundary falls outside of one of the images, modify the image such that the part of the boundary is interpolated therein.

7. The medical image processing apparatus according to claim 1, wherein the processor is configured to:
determine whether an artifact is shown in each of the images based on the segmentation data, and
upon determining that an artifact is shown in one of the images, modify the image such that a part of the image corresponding to the artifact is emphasized.

8. The medical image processing apparatus according to claim 1, wherein the processor is configured to:
input the acquired images into a second machine learning model that has been trained to detect a presence or an absence of an object in an image of a luminal organ, and for each of the acquired images, obtain information from the second machine learning model indicating whether or not an object is present or absent in the acquired image, and
superimpose an image of the object on the 3-D image based on the information.

9. The medical image processing apparatus according to claim 8, wherein the second machine learning model has been trained using a cross-sectional image of the luminal organ and data indicating whether an object exists on a scan line of the image at each angle.

10. The medical image processing apparatus according to claim 8, wherein the object includes at least one of an artifact and a lesion.

11. The medical image processing apparatus according to claim 8, wherein the object and the luminal organ are displayed in different colors.

12. The medical image processing apparatus according to claim 1, wherein the processor controls the catheter to acquire the images based on a gravity center moving distance of the luminal organ, predetermined cardiac cycle data, or correlation data of a predetermined index of the predetermined site.

13. A method carried out by a medical image processing apparatus for processing medical images of a luminal organ, the method comprising:
controlling a catheter having an ultrasonic probe to acquire a plurality of cross-sectional images of the luminal organ when the catheter is inserted into the luminal organ and moved along a longitudinal direction thereof;
inputting the acquired images into a first machine learning model that has been trained to classify each pixel in an image of a luminal organ and for each of the acquired images, obtaining position data indicating a boundary between predetermined regions of the luminal organ based on segmentation data output from the first machine learning model that classifies each pixel of the acquired image;
selecting two of the images that are consecutive and identifying a group of points corresponding to the boundary in each of the selected images based on the position data;
associating one or more of the points in one of the selected images with one or more of the points in the other image;
generating a 3-D image of the luminal organ in which said one or more of the points in one of the selected images are respectively connected to the associated points in the other image; and
displaying the generated 3-D image.

14. The method according to claim 13, wherein associating includes:
determining a difference between each of the points in one of the selected images and each of the points in the other image, and
associating one of the points in one of the selected images and one of the points in the other image, a difference of which is smallest.

15. The method according to claim 14, wherein one of the points in one of the selected images and one of the points in the other image, a difference of which is greater than or equal to a threshold, are not associated with each other.

16. The method according to claim 13, wherein in the 3-D image, said one or more of the points in one of the selected images are connected, and said one or more of the points in the other image are connected.

17. The method according to claim 13, further comprising:
determining whether a side branch of the luminal organ is shown in each of the images based on the segmentation data; and
determining not to connect one or more of the points in one of the selected images corresponding to the side branch and the associated points in the other image.

18. The method according to claim 13, further comprising:
determining whether a part of the boundary falls outside of each of the images based on the segmentation data; and
upon determining that a part of the boundary falls outside of one of the images, modifying the image such that the part of the boundary is interpolated therein.

19. The method according to claim 13, further comprising:
determining whether an artifact is shown in each of the images based on the segmentation data; and
upon determining that an artifact is shown in one of the images, modifying the image such that a part of the image corresponding to the artifact is emphasized.

20. A non-transitory computer readable medium storing a program causing a computer to execute a method for processing medical images of a luminal organ, the method comprising:
controlling a catheter having an ultrasonic probe to acquire a plurality of cross-sectional images of the luminal organ when the catheter is inserted into the luminal organ and moved along a longitudinal direction thereof;
inputting the acquired images into a first machine learning model that has been trained to classify each pixel in an image of a luminal organ and for each of the acquired images, obtaining position data indicating a boundary between predetermined regions of the luminal organ based on segmentation data output from the first machine learning model that classifies each pixel of the acquired image;
selecting two of the images that are consecutive and identifying a group of points corresponding to the boundary in each of the selected images based on the position data;
associating one or more of the points in one of the selected images with one or more of the points in the other image;
generating a 3-D image of the luminal organ in which said one or more of the points in one of the selected images are respectively connected to the associated points in the other image; and
displaying the generated 3-D image.
